(19) United States
(12) Patent Application Publication
(10) Pub. No.: US 2022/0313469 A1
(43) Pub. Date: Oct. 6, 2022

(54) LUMBAR SUPPORT BELT WITH SENSORS

(71) Applicant: THUASNE, LEVALLOIS PERRET (FR)

(72) Inventors: Clotilde SOUCHE, LYON (FR); Philippe MICHEL, CAPPELLE-EN-PEVELE (FR)

(21) Appl. No.: 17/616,108

(22) PCT Filed: Jun. 2, 2020
(86) PCT No.: PCT/EP2020/065214

§ 371 Date: Dec. 2, 2021

(30) Foreign Application Priority Data
Jun. 3, 2019 (FR) FR19 05838

(51) Int. Cl. A61F 5/02

(52) U.S. Cl. CPC A61F 5/028; A61F 2005/0188

(57) ABSTRACT

Disclosed is a lumbar support belt including: a support belt body intended to surround at least one part of a user's torso; at least a first sensor capable of measuring the pressure exerted by the support belt on a user or the elongation of the support belt body; and a module including at least a second sensor capable of measuring at least one parameter relating to the actimetry of the user. The module is removably attached to a second location of the support belt body.

### CROSS-REFERENCE TO RELATED APPLICATIONS

This application is the U.S. national phase of International Application No. PCT/EP2020/065214 filed Jun. 2, 2020 which designated the U.S. and claims priority to French Patent Application No. 19 05838 filed Jun. 3, 2019, the entire contents of each of which are hereby incorporated by reference.

### BACKGROUND OF THE INVENTION

### Field of the Invention

The present invention relates to a lumbar support belt comprising a support belt body adapted to surround the torso of a user and at least one sensor attached to the support belt body.

### Description of the Related Art

To monitor the use of the belt by a user and to provide feedback on said use, it is advantageous to provide sensors on the belt.

U.S. Pat. No. 9,839,553 B2 describes an orthopedic belt comprising a first pressure sensor placed on the abdomen, a second pressure sensor on the back and a posture detection sensor.

However, the placement of the sensors in relation to the belt is not precise.

The measurements made by the sensors are likely to be influenced by the integration of each sensor in relation to the belt.

In addition, such a belt does not allow for easy maintenance.

### SUMMARY OF THE INVENTION

An object of the invention is therefore to propose a lumbar support belt comprising sensors that make it possible to reproduce the measurement of each sensor and are easy to maintain.

To this end, the invention has as its object a lumbar support belt comprising:
a support belt body, intended to surround at least one part of the torso of a user,
at least a first sensor, capable of measuring the pressure exerted by the belt on a user or the elongation of the support belt body, and
a module comprising at least a second sensor, capable of measuring at least one parameter relating to the actimetry of the user, the module being removably attached to a second location of the support belt body.

The removability of the module facilitates the maintenance of the belt.

The placement of the sensors also makes it possible to reproduce the measurements, so that the measurements taken can be interpreted, as to the level of pressure exerted by the belt on a user.

The lumbar support belt may further have one or more of the following features, considered individually or in any technically possible combination:
the module comprises the at least one first sensor;
the module comprises an outer casing defining an inner volume, the at least one first sensor and the at least one second sensor being contained in the inner volume;
the support belt body is provided with a pocket, the module being capable of being inserted into the pocket;
the at least one first sensor is fixed to a ventral part of the support belt body intended to be placed against a ventral portion of a user's torso;
the support belt body comprises at least one elastic thread, the first sensor being able to measure the elongation of said elastic thread;
the support belt body is woven or knitted, the first sensor comprising at least one output terminal, the belt having a wire connection between the output terminal and the module, the wire connection comprising at least one conductive wire connected to said output terminal, the conductive wire being made with the support belt body;
the support belt body comprises at least one elastic portion, the conductive wire being elastic and made in the elastic portion of the support belt body;
the conductive wire is provided with an insulating coating, the conductive wire being connected at at least one connection point, a protective material being deposited locally at each connection point;
the module comprises a controller, the first sensor and the second sensor being connected to said controller; and/or
the module comprises a transfer unit, the controller being connected to the remote data transfer unit, such as a radio wave transmitter.

### BRIEF DESCRIPTION OF THE DRAWINGS

Further features and advantages of the invention will become apparent from the following description of embodiments of the invention, given by way of example only and with reference to the drawings in which:

FIG. 1 is a schematic view of a lumbar support belt according to a first embodiment of the invention,

FIG. 2 is a schematic view of a first sensor and an additional sensor of the lumbar support belt of FIG. 1,

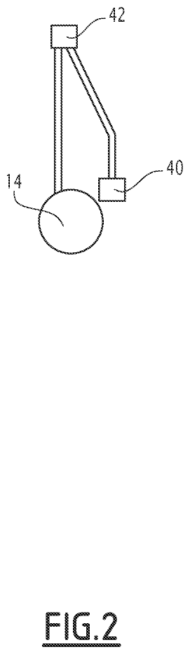

FIG. 3 is a schematic view of a module of the lumbar support belt of FIG. 1, and

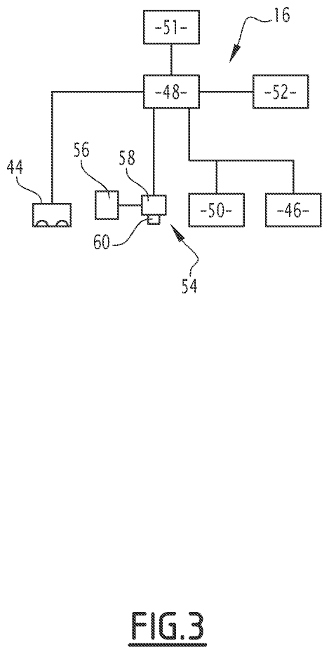

FIG. 4 is a schematic view of a lumbar support belt according to a second embodiment of the invention.

### DESCRIPTION OF THE PREFERRED EMBODIMENTS

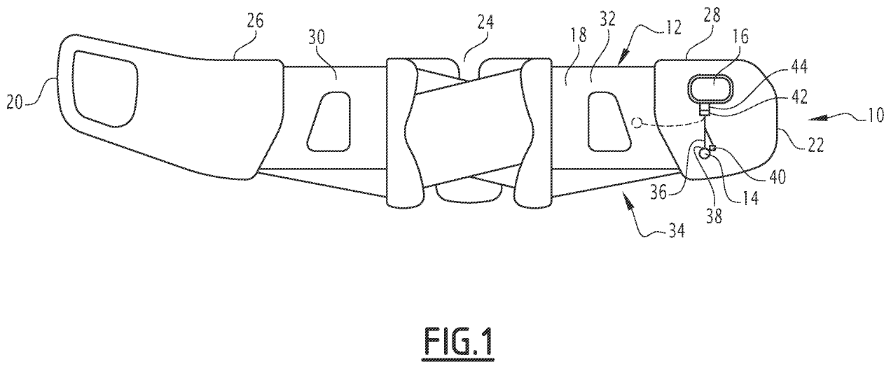

A lumbar support belt 10 according to a first embodiment of the invention is shown in FIG. 1.

The lumbar support belt 10 comprises a support belt body 12, intended to surround at least one part of a user's torso when the belt 10 is worn by that user, at least a first sensor 14, and a module 16.

The support belt body 12 is adapted to apply pressure to a user when surrounding the torso of said user, so as to support the use's torso, particularly to prevent or relieve back pain.

The support belt body 12 has an inner side not shown in FIG. 1 and intended to face the user's torso and an outer side 18 visible in FIG. 1 and opposite the inner side.

The support belt body 12 extends between a first end 20 and a second end 22, the first end 20 and the second end 22 being reversibly attached to each other when the support belt body 12 is worn by a user.

The first end 20 and the second end 22 are, for example, each provided with at least one complementary hook-and-loop Velcro type of strip on the inner side, at one end, and on the outer side 18, at the other end, the hook-and-loop Velcro type of strips interacting with each other when the belt is closed around the use's torso.

The support belt body 12 comprises a dorsal portion 24 adapted to extend against a use's spine when the belt is worn, two ventral portions 26, 28 adapted to extend against a ventral portion of a user's torso when the belt is worn, and two intermediate portions 30, 32 connecting the dorsal portion and one of the respective ventral portions. The first and second ends 20 and 22 each extend adjacent to one of the free ends of the ventral portions.

Here, the support belt body 12 is woven or knitted.

The support belt body 12 comprises at least one elastic portion 34.

Here, “elastic” means that the support belt body 12 in said portion is capable of being stretched by at least 10%, more particularly by at least 20% and advantageously between 55% and 65%, without damage, and capable of recovering its initial dimension in its elastic area, in the absence of a stretching force.

The elastic portion 34 is located in one of the intermediate portions 32, of the support belt body 12.

The support belt body 12 comprises at least one elastic thread, more particularly in the elastic portion. The elastic portion is, for example, formed substantially of elastic threads.

In a variant, the support belt body 12 does not comprise an elastic portion or an elastic thread.

The first sensor 14 is capable of measuring the pressure exerted by the belt 10 on a user or the elongation of the support belt body 12, more particularly the stretching of the support belt body 12 in the elastic portion 34.

The first sensor 14 is furthermore sealed or encapsulated in a waterproof manner within the support belt body 12 such that it can be washed off the belt without damage.

The first sensor 14 comprises at least one output terminal.

In the embodiment shown in the solid line, the first sensor 14 is attached to one of the ventral portions of the support belt body 12.

Here, the ventral portion is not elastic.

The first sensor 14 is a capacitive pressure sensor, for example, i.e. it has a capacitance that increases when pressure is applied to the sensor 14, said capacitance being measured to calculate the corresponding pressure.

In a variant, the first sensor 14 is a resistive pressure sensor.

In the embodiment shown as a dashed line, the first sensor 14 is attached to an intermediate portion 32, more particularly in the elastic portion 34.

Additionally or In a variant, the first sensor 14 is capable of measuring the elongation of a wire or one part of the belt. “Elongation of the belt” then means the elongation of said wire or said belt portion.

The first sensor 14 is capable of measuring the elongation in the elastic portion, for example, more particularly of one of the elastic threads forming the elastic portion.

The first sensor 14 is an inductive elongation sensor or a resistive elongation sensor, for example, based on an elastic optical fiber incorporating conductors as described in WO 2017137945 A1 or WO2014047660 A1.

The first sensor 14 is immovably attached to a first location of the support belt body 12.

The first location is a fixed, predetermined location of the support belt body 12.

This allows for accurate positioning of the first sensor 14 in relation to the belt, and thus the positioning of the first sensor 14 in relation to the user when the user tightens the belt with the same level of tightness, thereby ensuring consistency of measurements from one measurement to another.

This facilitates a more suitable interpretation, in particular, of the measurements of the first sensor 14.

It is integrated with said support belt body 12, for example.

“Integrated” means that the first sensor is incorporated into the support belt body 12. It is inserted into the fabric or knitted fabric forming the support belt body 12, for example. In a variant, the first sensor 14 is not inserted into the fabric or knit fabric, but the set of connecting wires of said first sensor 14.

In the embodiment shown in FIG. 1, the first sensor is embedded in the elastic portion 34 of the support belt body 12. The first sensor 14 here is capable of measuring the pressure exerted by the belt 10 on a user or the elongation of the support belt body 12.

In a variant embodiment, the first sensor 14 is integrated into a non-elastic or even rigid portion of the belt. The first sensor 14 is then capable of measuring the pressure exerted on the user by the belt 10.

The first sensor 14 is integrated in a board also comprising an integrated circuit or wires provided to make a connection with the module, for example. The board is waterproof, more particularly made of plastic, and is integrated in the support belt body 12.

The belt 10 advantageously comprises an additional temperature sensor 40 capable of measuring the temperature of the first sensor 14 or around it. This makes it possible to adjust the measurement of the first sensor 14 based on the temperature, in particular, in cases where a temperature variation would be likely to cause a drift in the measurements made by the first sensor 14.

In a variant, the first sensor 14 is not sensitive to temperature, at least in one usage range. The belt 10 does not comprise an additional temperature sensor 40 capable of measuring the temperature of or around the first sensor 14.

The belt is associated with a unique identifier, for example, stored in a medium such as a radio frequency identification (RFID) or near field communication (NFC) chip, for example. The unique identifier comprises an indication of the belt model, with the controller being able to read said identifier within the chip.

The unique identifier support is attached to the first sensor 14.

The module 16 is removably attached to a second location on the support belt body 12. The second location on the support belt body 12 is fixed to the belt 10, i.e., the module 16 is fixed at the same point when it is reattached to the support belt body 12 after being removed therefrom.

The module 16 is attached near the first sensor 14, specifically in the elastic portion 34 of the support belt body 12.

The module 16 is removably attached to one of the ventral portions 28 of the support belt body 12.

The module 16 is formed of a shell, for example, defining an inner volume, with all of the elements of the module 16 described below being included in the inner volume.

The module 16, more particularly the shell, has a connection interface 44, provided for electrically connecting the module to a corresponding terminal 42 of the support belt body 12. Said terminal forms the fixed attachment location of the module 16.

The module 16 comprises at least a second sensor 46, capable of measuring at least one parameter relating to the actimetry of the user.

The precise positioning of the second sensor 46 is known in relation to the belt, with the module 16 fixed at a given location, and thus the positioning of the second sensor 46 with in relation to the user when the user tightens the belt with the same level of tightness.

This facilitates very accurate interpretation of the user's movements, for example.

The second sensor 46 is a motion sensor, for example, operating along three axes, i.e., capable of measuring motion along three axes forming a space, or a gyroscope.

The actimetry parameter is a number of steps taken by the user wearing the belt 10, for example.

The module 16 further comprises a controller 48, more particularly a microprocessor capable of processing the data transmitted by the first sensor 14, the second sensor 46 and the additional temperature sensor 40 by software.

The module 16 further comprises a memory 50 connected to the controller 48 such that the controller 48 has access to said memory 50 to store and/or retrieve data therefrom.

The module 16 further comprises a clock 51 associated with the controller 48. The clock 51 is adapted to provide a time stamp at any time.

The second sensor 46 is connected to the controller 48, so as to allow transmission of data from the second sensor 14 to said controller 20.

The second sensor 46 is connected to said controller 48, more particularly in a wired manner directly within the module.

In a variant, the second sensor 46 is connected to the controller 48 in a non-wireless manner, by a wireless communication means such as radio waves or near field communication (or NFC), for example.

The controller 48 is further connected to a remote data transfer unit 52.

The transfer unit 52 is capable of emitting a signal comprising data transmitted by the controller 48.

The transfer unit 52 is connected to the controller 48 by a wire connection within the module 16, for example.

The transfer unit 52 is a radio wave transmitter, for example. It follows the Wi-Fi™ protocol or the Bluetooth® standard, for example.

In a variant, the transfer unit 52 uses Near Field Communication (NFC).

In particular, the transfer unit 52 allows data to be transferred from the controller 48 to an outer computer, for example, to allow feedback to the user or patient monitoring.

The module 16 further comprises a battery system 54 connected to the controller 48, advantageously in a wired manner within the module 16.

The battery system 54 here comprises a rechargeable battery 56 and a charger 58.

The battery 56 is, for example, a lithium-ion battery.

The charger 58 comprises a charging interface 60 adapted to be connected to a source of electricity, such as a power grid, to recharge the battery 56 via the charger 58.

The charging interface 60 is a micro-USB connection, for example.

The belt 10 has an interruptible wire connection between the output terminal of the first sensor 12 and the module 16.

The wire connection comprises at least one conductive wire 36 having a first end connected to said output terminal and a second end provided with a connection terminal 42.

The connection terminal 42 corresponds to the attachment terminal of the module 16 described above.

The connection terminal 42 is complementary to the connection interface 44 of the module 16. The connection interface 44 of the module 16 is removably attached to the connection terminal 42, so as to form the wire connection between the first sensor and the module.

The conductive wire 36 in the embodiment shown is elastic and integrated directly into the elastic fabric or knit fabric of the support belt body, i.e. the elastic conductive wire 36 is woven or knitted directly with the wires constituting the support belt body 12.

In a variant, the conductive wire 36 is sewn, woven, or added to the support belt body after the support belt body 12 is made.

The conductive wire 36 is said to be made together with the support belt body 12.

In a variant, the conductive wire 36 is not elastic.

The elastic conductive wire 36 has a resistance of between 5 ohms and 20 ohms per meter of wire.

The elastic conductive wire 36 has an overall titer of between 730 dtex and 2460 dtex. In particular, this allows the elastic conductive wire to be knitted or woven with the support belt body 12 on a standard machine.

The elastic conductive wire 36 comprises an elastic core wire, for example, at least one electrically conductive covered wire wrapped by covering around the elastic core wire, and at least one non-conductive wire covering the conductive covered wire.

The non-conductive wire covers the conductive covered wire completely, for example.

The non-conductive wire is also wrapped around the elastic core wire, more particularly in the opposite direction to the conductive wrapped wire.

The elastic core wire has a double wrap.

The conductive covered wire is made of copper and/or silver. More particularly, it is a copper wire surrounded by a silver layer.

The conductive covered wire has a diameter of between 30 μm and 100 μm. It has one or more conductive filaments.

The conductive covered wire is advantageously varnished with an insulating varnish. This makes it possible to seal the conductive covered wire and protect it from physical damage or oxidation.

The conductive covered wire has a number of turns per meter between 600 and 1700. This facilitates an optimal conduction of electricity.

The conductive covered wire has an elongation at the break strictly greater than 60%, here more particularly strictly greater than 80%.

The elastic conductive wire 36 is connected at the connection point(s) 38, in particular at the output terminal of the first sensor 14 and at the connection terminal 42. At each connection point, the conductor wire is free of varnish, with a protective material being applied locally at each connection point. The protective material is silicone, for example.

In particular, this makes it possible to seal off the entire assembly at the connection points. This is advantageous for allowing the belt to be washed without damaging the electrical connections.

Advantageously, the additional temperature sensor 40 is also connected to the connection terminal 42, advantageously by an elastic conductive wire similar to that connecting the first sensor to the connection terminal 42, so as to be reversibly fixed to the connection interface of the module 18.

Thus, the first sensor 14 and the additional temperature sensor 40 are each connected to the controller 48 of the module 16, so as to allow data transmission from each of said sensors 14, 40 to said controller 48, more particularly via the connection interface 44 of the module 16.

In a variant, the first sensor 14 and/or the additional temperature sensor 40 are connected to the controller 48 in a non-wireless manner, such as by a wireless communication means such as radio waves or near field communication (NFC).

The integration of the first sensor 14 in the belt 10 and the structure provided for the connection of the module 16 notably make the support belt body 12 with the integrated first sensor 14 machine washable. For example, the support belt body 12 with the first sensor 14 withstands a number of washes at a temperature of 30° C. strictly greater than 15.

The placement of the second sensor in a removable module further facilitates belt maintenance. Indeed, it is enough to remove the module before washing the belt. Thus, there is no risk of damaging the module and the second sensor placed inside during the washing of the belt.

In an embodiment not shown, the lumbar support belt further comprises an additional band provided to wrap a user's torso around the support belt body 12 in order to apply additional compression.

The integration of the first sensor on or within the support belt body allows for a fixed placement of said sensor.

The measurements taken can be reproduced, so that the measurements as to how tight the belt is on the user can be interpreted.

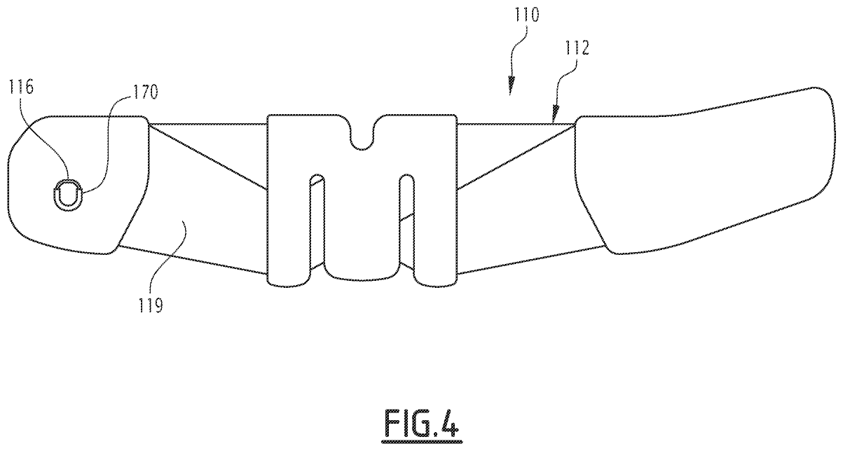

A belt 110 according to a second embodiment is shown in FIG. 4, and only those features in which the second embodiment differs from the first embodiment are described here.

The same or similar features have a reference equal to that of the first embodiment incremented by 100.

The support belt body 112 is provided with a pocket 170.

The pocket 170 extends from the inner side 119 of the support belt body 112.

The pocket 170 comprises a textile, a fabric here defining an inner volume of the pocket 170.

The pocket 170 has a single opening providing access to said inner volume of the pocket at a top edge of the textile.

The perimeter of the textile outside of the upper edge is integral with, for example sewn to the support belt body 112.

The perimeter comprises a lower edge, the upper edge and two sides each connecting the lower edge and the upper edge.

Advantageously, the pocket 170 is provided with a tab attached to the textile. The tab makes it possible to grip the textile so as to move the textile away from the support belt body 112, to facilitate access to the inner volume of the pocket.

The textile has an elasticity of between 20% and 200%. It is made of Lycra, for example.

In a variant, the textile is not elastic. The pocket defines an inner volume complementary to the module 116.

In this second embodiment, the first sensor is not attached to one of the ventral portions of the support belt body, but is integrated into the module 116.

The first sensor is a capacitive or resistive pressure sensor, for example.

The module 116 comprises an outer case defining an inner volume, with the at least one first sensor and the at least one second sensor included within the inner volume.

The controller, memory, transfer unit, and battery system as described with respect to the first embodiment are similarly included in the outer case of the module 116.

Here, the first sensor is directly connected to the controller 48 by a wire connection within the module 116.

The module 116, specifically the outer case, does not have a connection interface provided to electrically connect the module to a corresponding terminal on the support belt body.

The electronics assembly is contained within the module 116.

The integration of all the electronics into the removable module 116 allows the support belt body 112, in particular, to be made machine washable by simply removing the module 116.

The module 116 is capable of being inserted, here by the upper edge, in a removable manner into the pocket 170, more particularly into the inner volume of the pocket 170, corresponding here to the second location of the support belt body 12.

The pocket 170 is sized such that the module 116 inserted into the pocket 170 is held stationary in relation to the support belt body 112 by the textile of the pocket 170.

More particularly, the module 116 is arranged to be inserted into the pocket 170 with less than 0.1 mm of play, preferably no play.

Furthermore, the elasticity of the textile improves the holding of the module 116 by the pocket 170 against the support belt body 112, more particularly against the inner face 119.

Thus, the second location of the support belt body 112 is fixed on the belt 110.

The precise positioning of the module 116 within the pocket 170 allows for precise positioning of the first sensor and the second sensor.

This allows for accurate positioning of the first sensor and the second sensor in relation to the belt, and thus the positioning of the first sensor and the second sensor in relation to the user when the user tightens the belt with the same level of tightness, thereby ensuring consistency of measurements from one measurement to another.

In particular, this enables a more appropriate interpretation of the measurements of the first and second sensors.

The measurements taken can be reproduced, so that they can be interpreted.

In a variant embodiment similar to the first embodiment, the module 16 described in connection with the first embodiment is attached to the belt by insertion into a pocket as described with respect to the second embodiment.

A lumbar support belt according to the invention thus makes it possible to reproduce the measurement of each sensor and is easy to maintain.

In particular, such a lumbar support belt 10, 110 makes it possible to monitor the wearing of the belt, in particular via a method for determining the wearing of the belt by a user.

An example embodiment of the method comprises the following steps:
providing a belt as described above,
acquiring first data by the at least one first sensor 14,
acquiring second data by the at least one second sensor 46, and
simultaneously processing the first and second data to determine whether the belt is being worn.

During the first data acquisition step, the at least one first sensor 14 acquires first data corresponding to the pressure applied by the belt on a user or the elongation of the belt, as described above.

Measurements are taken by the first sensor 14 at regular intervals.

In a variant, the first sensor 14 acquires measurements continuously.

The first data is then transmitted to the controller 18.

The first data may undergo data preprocessing before or during transmission. In this case, the first transmitted data is defined as the first data preprocessed and transmitted.

In a variant, the preprocessing is performed after transmission by the controller 48.

The pre-processing corresponds to a de-noising, for example, in particular by frequency filtering.

During the second data acquisition step, the at least one second sensor 46 acquires second data corresponding to at least one parameter relating to the user's actimetrics, as described above.

Measurements are taken by the second sensor 46 at regular intervals, at a frequency of 25 Hz, for example.

In a variant, the second sensor 46 acquires measurements continuously.

The second data is then transmitted to the controller 48.

The second data may undergo data preprocessing before or during transmission. In this case, the transmitted second data is defined as the second data preprocessed and transmitted.

In a variant, the one or multiple preprocessing is performed after transmission by the controller 48.

The pre-processing corresponds to a denoising, for example, in particular by frequency filtering.

The first and second transmitted data is then processed.

The simultaneous data processing step is carried out here by the controller 48.

It takes into account the belt model, for example, to allow a more reliable detection of the data by the sensors in relation to the belt.

The controller 48 initially considers that the orthosis is not worn, for example.

If a first and second wearing condition are concurrently met, then it is determined that the belt is worn.

The first wearing condition here is that the pressure or elongation measured by the first sensor for a first period greater than a first minimum period is greater than a first predetermined value Min P.

The first predetermined value Min P corresponds here to a minimum pressure or a minimum elongation.

The second wearing condition here is that the actimetry-related parameter is greater than a second predetermined value during a second period greater than a second minimum period Tobs.

The second predetermined value corresponds to a minimum value of movement on each of the axes.

The first period is included in the second period or the second period is included in the first period.

Here, the first period and the second period coincide.

When the wearing of the orthosis is detected when previously the orthosis was indicated as not worn, the controller records an event corresponding to the beginning of the wearing of the orthosis, to which a date provided by the inner clock is associated.

Furthermore, if it is determined, during the step of simultaneous data processing, that the belt is worn, then the method further comprises a step of determining an orthosis tightening level.

When the belt is previously detected as being worn, but one of the first and second removal conditions occurs, then it is detected that the belt is no longer worn.

The first condition here is that the pressure or elongation measured by the first sensor is less than a first predetermined value Min P for a first period greater than a first minimum period.

The first period and the first predetermined value Min P here are identical to those of the first condition.

The second condition here is that the actimetry-related parameter is less than a second predetermined value during a second period greater than a second minimum period Tobs.

The second period Tobs and the second predetermined value here are identical to those of the first condition.

The controller then records an event corresponding to the end of the wearing the belt, to which a date provided by the inner clock is related.

Following the processing step, the method advantageously comprises a step of transmitting at least one result of the step of simultaneous data processing of the controller of the module 16 to an outer computer by the transfer unit 52.

The at least one result transmitted to the external computer comprises the clamping level.

The outer computer is capable of displaying the at least one transmitted result to make follow-up possible, for example, for the user and/or a medical practitioner following said user.

Advantageously, the outer computer calculates additional information from the at least one result transmitted by the transfer unit 52, in particular the belt wearing time.

The use of two sensor types and simultaneous data processing facilitate more reliability in detecting the wearing of the orthosis by comparing the data obtained as well as, advantageously, data specific to the belt model, and by detecting the situation at a given moment in a more reliable way.

This makes it possible to have better data for the medical follow-up of a patient or a better feedback to a user.

Repeating the measurements makes it possible to check treatment compliance. Furthermore, the data obtained by the belt can be linked to other information such as pain or discomfort felt by the patient, to know if the belt is effective.

1. A lumbar support belt comprising:
a support belt body for surrounding at least one part of a user's torso,
at least a first sensor capable of measuring the pressure exerted by the belt on a user or the elongation of the support belt body, and
a module comprising at least a second sensor adapted to measure at least one parameter relating to the actimetry of the user, the module being removably attached to a second location of the support belt body.

2. The lumbar support belt according to claim 1, wherein the module comprises the at least one first sensor.

3. The lumbar support belt according to claim 2, wherein the module comprises an outer casing defining an inner volume, the at least one first sensor and the at least one second sensor being contained in the inner volume.

4. The lumbar support belt according to claim 1, wherein the support belt body is provided with a pocket, the module being capable of being inserted into the pocket.

5. The lumbar support belt according to claim 1, wherein the at least one first sensor is attached to a ventral portion of the support belt body for placement against a ventral portion of the user's torso.

6. The lumbar support belt according to claim 1, wherein the support belt body comprises at least one elastic thread, the first sensor being capable of measuring the elongation of said elastic thread.

7. The lumbar support belt according to claim 1, wherein the support belt body is woven or knitted, the first sensor comprising at least one output terminal, the belt having a wire connection between the output terminal and the module, the wire connection comprising at least one conductive wire connected to said output terminal, the conductive wire being made with the support belt body.

8. The lumbar support belt according to claim 7, wherein the support belt body comprises at least one elastic portion, the conductive wire being elastic and formed into the elastic portion of the support belt body.

9. The lumbar support belt according to claim 7, wherein the conductive wire is provided with an insulating varnish, the conductive wire being connected at at least one connection point, a protective material being deposited locally at each connection point.

10. The lumbar support belt according to claim 1, wherein the module comprises a controller, the first sensor and the second sensor being connected to said controller.

11. The lumbar support belt according to claim 10, wherein the module comprises a transfer unit, the controller being connected to the remote data transfer unit, such as a radio wave transmitter.

12. The lumbar support belt according to claim 2, wherein the support belt body is provided with a pocket, the module being capable of being inserted into the pocket.

13. The lumbar support belt according to claim 3, wherein the support belt body is provided with a pocket, the module being capable of being inserted into the pocket.

14. The lumbar support belt according to claim 2, wherein the at least one first sensor is attached to a ventral portion of the support belt body for placement against a ventral portion of the user's torso.

15. The lumbar support belt according to claim 3, wherein the at least one first sensor is attached to a ventral portion of the support belt body for placement against a ventral portion of the user's torso.

16. The lumbar support belt according to claim 4, wherein the at least one first sensor is attached to a ventral portion of the support belt body for placement against a ventral portion of the user's torso.

17. The lumbar support belt according to claim 2, wherein the support belt body comprises at least one elastic thread, the first sensor being capable of measuring the elongation of said elastic thread.

18. The lumbar support belt according to claim 3, wherein the support belt body comprises at least one elastic thread, the first sensor being capable of measuring the elongation of said elastic thread.

19. The lumbar support belt according to claim 4, wherein the support belt body comprises at least one elastic thread, the first sensor being capable of measuring the elongation of said elastic thread.

20. The lumbar support of claim 11, wherein the transfer unit is a radio transmitter.
